(19) United States
(12) Patent Application Publication
(10) Pub. No.: US 2019/0118120 A1
(43) Pub. Date: Apr. 25, 2019

(54) SAMPLING FILTER, SAMPLING DEVICE, AND SAMPLING METHOD USING THE SAME

(71) Applicant: Murata Manufacturing Co., Ltd., Nagaokakyo-shi (JP)

(72) Inventors: Masaru Banju, Nagaokakyo-shi (JP); Junko Watanabe, Nagaokakyo-shi (JP); Takashi Kondo, Nagaokakyo-shi (JP)

(21) Appl. No.: 16/223,627

(22) Filed: Dec. 18, 2018

Related U.S. Application Data
(63) Continuation of application No. PCT/JP2017/026682, filed on Jul. 24, 2017.

(30) Foreign Application Priority Data
Aug. 30, 2016 (JP) 2016-167713

(51) Int. Cl. B01D 29/03; B01D 29/56; B01D 39/10; G01N 1/40; C12M 1/00

(52) U.S. Cl. CPC B01D 29/03; B01D 29/56; B01D 39/10; G01N 2001/4088; C12M 47/02; B01D 2201/184; B01D 2201/188; G01N 1/4077

(57) ABSTRACT

A sampling filter and a sampling device that can readily sample cell aggregates having a predetermined size are provided, and a sampling method using the same is provided. A sampling filter samples cell aggregates and includes a frame portion that demarcates a region through which a liquid containing cell aggregates passes and a filter portion which is disposed in only part of the region and in which a plurality of through holes are located.

### CROSS REFERENCE TO RELATED APPLICATIONS

The present application is a continuation of International application No. PCT/JP2017/026682, filed Jul. 24, 2017, which claims priority to Japanese Patent Application No. 2016-167713, filed Aug. 30, 2016, the entire contents of each of which are incorporated herein by reference.

### TECHNICAL FIELD

The present invention relates to a sampling filter, a sampling device, and a sampling method using the same.

### BACKGROUND ART

Japanese Unexamined Patent Application Publication No. 2009-180594 (Patent Document 1) discloses a sampling device that samples the state of cells immersed in a culture medium.

The sampling device described in Patent Document 1 has a problem in that cell aggregates having a predetermined size are not readily sampled.

In recent years, when cell aggregates or the like to be used for examining drug efficacy are cultured, it is required that cell aggregates having a predetermined size be cultured in order to reduce variations in drug efficacy data. Therefore, in order to grasp the culture state of the cell aggregates having the predetermined size, sampling of the cell aggregates having the predetermined size has been required. However, in the device described in Patent Document 1, sampled cell aggregates contain cell aggregates having different sizes because sampling is performed by delivering the culture medium from a culture container to the outside through a culture medium delivery pipe.

As a result, in order to sample cell aggregates having a predetermined size from the culture medium using this sampling device, filtering has to be performed at least twice. Specifically, a first filtering is carried out to remove cell aggregates having a size larger than the predetermined size from the culture medium and then a second filtering is carried out to remove cell aggregates having a size smaller than the predetermined size. As a result, in order to sample cell aggregates having the predetermined size from the culture medium, a plurality of steps have to be performed and this requires a lot of time and effort.

It is an object of the present invention to provide a sampling filter and a sampling device that can readily sample cell aggregates having a predetermined size and to provide a sampling method using the same.

### BRIEF DESCRIPTION OF THE INVENTION

The present inventors performed intensive research and found a sampling filter including a filter portion in part of the region through which a liquid containing cell aggregates passed, the filter portion having a plurality of through holes.

A sampling filter according to an aspect of the present invention is a sampling filter that samples cell aggregates and that includes a frame portion that demarcates a region through which a liquid containing cell aggregates passes. The sampling filter further includes a filter portion which is disposed in only part of the region and in which a plurality of through holes are located.

According to such a configuration, cell aggregates having a predetermined size can be readily sampled.

In the sampling filter, the proportion of the area of the filter portion may be between 1% and 10% (i.e., 1% or more and 10% or less) of the area of the region when viewed from a fist principal surface of the sampling filter.

According to such a configuration, an increase in the passage resistance of the liquid due to the filter portion can be suppressed.

In the sampling filter, the plurality of through holes in the filter portion may include first through holes and second through holes smaller than the first through holes.

According to such a configuration, cell aggregates having different sizes can be readily sampled.

In the sampling filter, the filter portion may be disposed along an inner wall of the frame portion.

According to such a configuration, cell aggregates can be readily sampled from the liquid that flows along the inner wall of the frame portion.

The sampling filter may include a holding portion that extends from the frame portion and that holds the filter portion.

According to such a configuration, the strength of the filter portion can be enhanced by holding the filter portion by the holding portion. In addition, the filter portion can be arranged at any place in the region, through which the liquid passes, by holding the filter portion by the holding portion.

A primary component of the sampling filter may be at least one of a metal or a metal oxide.

According to such a configuration, the mechanical strength of the sampling filter can be enhanced.

A sampling device according to an aspect of the present invention includes a container portion having an inflow port that introduces a liquid containing cell aggregates and an outflow port that discharges the liquid and a sampling filter arranged between the inflow port and the outflow port of the container portion. The sampling filter includes a frame portion that demarcates a region through which the liquid containing cell aggregates passes and a filter portion which is disposed in part of the region and in which a plurality of through holes are located.

According to such a configuration, cell aggregates having a predetermined size can be readily sampled.

In the sampling device, the proportion of the area of the filter portion may be between 1% and 10% of the area of the region when viewed from a fist principal surface of the sampling filter.

According to such a configuration, an increase in the passage resistance of the liquid due to the filter portion can be suppressed.

In the sampling device, the plurality of through holes of the filter portion may include first through holes and second through holes smaller than the first through holes.

According to such a configuration, cell aggregates having different sizes can be readily sampled.

In the sampling device, the filter portion may be disposed along an inner wall of the frame portion.

According to such a configuration, cell aggregates can be readily sampled from the liquid that flows along the inner wall of the frame portion.

In the sampling device, the sampling filter may include a holding portion that extends from the frame portion and that holds the filter portion.

According to such a configuration, the strength of the filter portion can be enhanced by holding the filter portion by the holding portion. In addition, the filter portion can be arranged at any place in the region, through which the liquid passes, by holding the filter portion by the holding portion.

In the sampling device, a primary component of the sampling filter may be at least one of a metal or a metal oxide.

According to such a configuration, the mechanical strength of the sampling filter can be enhanced.

The sampling device may further include a filtration filter which is nearer than the sampling filter to the inflow port of the container portion and in which a plurality of through holes are located,

wherein the through holes in the filtration filter may be larger than the through holes in the sampling filter.

According to such a configuration, cell aggregates having a size larger than a predetermined size can be efficiently filtrated by the filtration filter and, in addition, cell aggregates having a predetermined size can be readily sampled.

In the sampling device, the filtration filter may trap first cell aggregates, and the sampling filter may trap second cell aggregates smaller than the first cell aggregates.

According to such a configuration, first cell aggregates having a size larger than a predetermined size can be trapped by the filtration filter and, in addition, second cell aggregates having a predetermined size can be readily trapped by the sampling filter.

The sampling device may include a plurality of sampling filters. The plurality of sampling filters can include a first sampling filter arranged between the inflow port and the outflow port of the container portion and a second sampling filter disposed nearer than the first sampling filter to the outflow port of the container portion. Third through holes which are smaller than the first through holes in the first sampling filter are located in the second sampling filter.

According to such a configuration, cell aggregates having different sizes can be readily sampled.

A sampling method according to an aspect of the present invention includes preparing or obtaining a sampling filter including a frame portion that demarcates a region through which a liquid containing cell aggregates passes and a filter portion which is disposed in part of the region and in which a plurality of through holes are located. Liquid containing cell aggregates is then passed through the sampling filter.

According to such a configuration, cell aggregates having a predetermined size can be readily sampled.

The sampling method may further includes preparing a filtration filter which is arranged upstream of the sampling filter and in which a plurality of through holes are located, and passing the liquid containing cell aggregates through the filtration filter so as to trap first cell aggregates. The modified liquid passing through the filtration filter includes second cell aggregates which are smaller than the first cell aggregates. The modified liquid is then passed through the sampling filter.

According to such a configuration, first cell aggregates having a size larger than a predetermined size can be trapped by the filtration filter and, in addition, second cell aggregates having a predetermined size can be readily trapped by the sampling filter.

In the sampling method, first and second sampling filters may be used. In this case, the liquid containing cell aggregates can be first passed through the first sampling filter so as to trap second cell aggregates of a second size and then passed through the second sampling filter to trap third cell aggregates of a third size which is smaller than the size of the second cell aggregates.

According to such a configuration, cell aggregates having different sizes can be readily sampled.

The filtration filter according to the present invention can readily sample cell aggregates having a predetermined size. Therefore, the present invention is useful for application to sampling of cell aggregates having a predetermined size from a culture medium

### BRIEF DESCRIPTION OF THE DRAWINGS

FIG. 1 is a schematic diagram showing a sampling filter in a first embodiment according to the present invention.

FIG. 2 is a magnified perspective view showing part of a filter portion in the sampling filter shown in FIG. 1.

FIG. 3 is a schematic diagram showing part of the filter portion in the sampling filter shown in FIG. 2, when viewed in the thickness direction.

FIG. 4 is a schematic diagram showing a sampling device in the first embodiment according to the present invention.

FIG. 5 is a schematic diagram showing a sampling filter in a modified example of the first embodiment according to the present invention.

FIG. 6 is a schematic diagram showing a sampling filter in another modified example of the first embodiment according to the present invention.

FIG. 7 is a schematic diagram showing a sampling filter in another modified example of the first embodiment according to the present invention.

FIG. 8 is a schematic diagram showing a sampling filter in another modified example of the first embodiment according to the present invention.

FIG. 9 is a schematic diagram showing a sampling device in a second embodiment according to the present invention.

FIG. 10 is a schematic diagram showing a filtration filter used in the sampling device in the second embodiment according to the present invention.

FIG. 11 shows a sampling device in a modified example of the second embodiment according to the present invention.

### DETAILED DESCRIPTION OF THE PREFERRED EMBODIMENTS

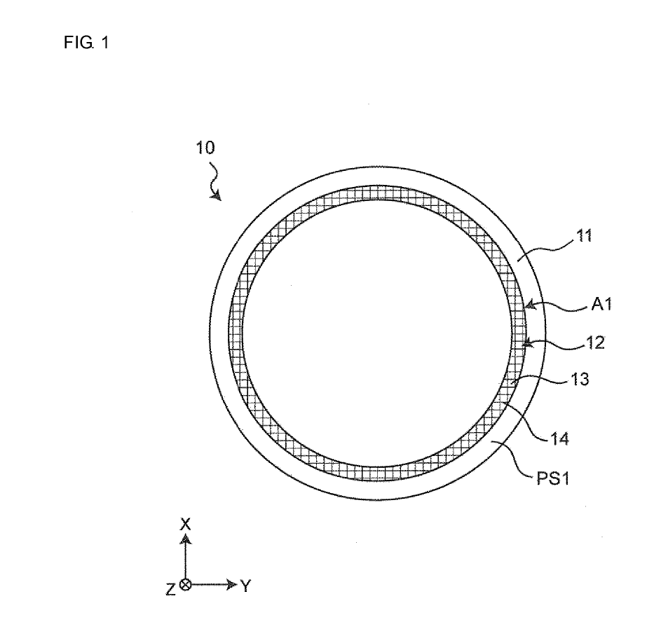

Referring now to the drawings wherein like numerals indicate like elements, there is shown in FIGS. 1-5 a first embodiment according to the present invention. In each drawing (of each of the disclosed embodiments), elements may be exaggerated for the sake of facilitating illustration.

[Sampling Filter]

FIG. 1 is a schematic diagram showing a sampling filter 10 in accordance with a first embodiment. The X-axis, the Y-axis, and the Z-axis in FIG. 1 indicate the vertical direction, the horizontal direction, and the thickness direction, respectively, of the sampling filter (as viewed in the orientation of the filter shown in FIG. 1).

The sampling filter 10 includes a frame portion 11 whose inner periphery demarcates a region A1 through which a liquid containing cell aggregates passes and a filter portion 12 disposed in part of the region A1.

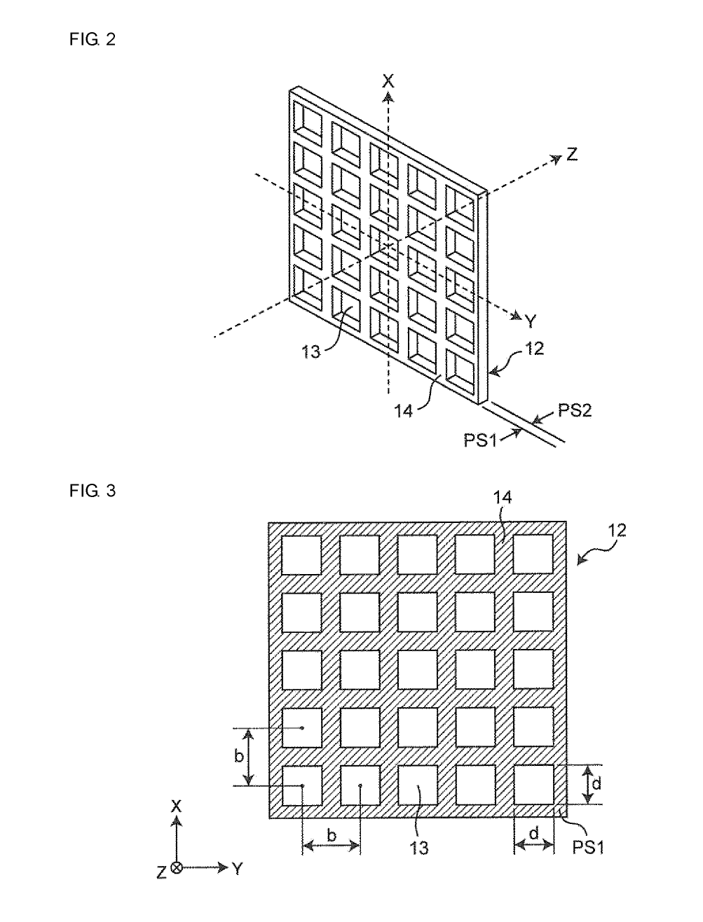

FIG. 2 is a magnified perspective view showing part of a filter portion 12 in the sampling filter 10 shown in FIG. 1. As shown in FIG. 2, a plurality of through holes 13 are located in the filter portion 12. Each of the through holes pass through first and second opposed principal surfaces PS1 and PS2 of the sampling filter 10.

The sampling filter 10 includes a filter portion 12 (only a part of the region A1) that captures cell aggregates contained in a culture medium (liquid) that passes through the region A1 inside the frame portion 11 so as to sample cell aggregates having a predetermined size.

As used herein, the phrase “cell aggregate” refers to aggregate of cells formed by a plurality of cells adhering to each other. Examples of cells constituting the aggregate include cancerous cells, stem cells, induced pluripotent stem cells (ips cells), ES cells, mesenchymal stem cells, and regenerative medicine cells. In the present specification, the liquid is, for example, a culture medium including an amino acid, protein, serum, and the like, phosphate buffered saline, or water. The liquid containing cell aggregates may contain substances derived from non-living materials, for example, resin particles, some of tissues of pieces of bone, pieces of meat, or the like, or dead cells in addition to the cell aggregates and the liquid.

In the first embodiment, the sampling filter 10 is in the shape of, for example, a circle. By way of example, the sampling filter 10 may have a diameter of 8 mm and the thickness of 15 μm. The primary component of the sampling filter 10 is preferably at least one of a metal and a metal oxide. The material constituting the sampling filter 10 may be, for example, gold, silver, copper, platinum, nickel, palladium, an alloy thereof, or an oxide thereof. While the disclosed embodiment of filter 10 is shown as circular in shape, it can, for example, have a rectangular shape such as a rectangle or a square, or may have an elliptical shape or the like.

<Frame Portion>

The frame portion 11 is formed so as to demarcate a region A1 through which a liquid containing cell aggregates passes (in this embodiment, the portion of the filter located radially inward of the inner periphery of the frame portion 11). The frame portion 11 is preferably formed in the shape of a ring when viewed from the first principal surface PS1. The frame portion 11 need not be solid and may have a number of through holes 13 per unit area which is less than that of the filter portion 12. The number of through holes 13 in the frame portion 11 is preferably not greater than 1% of the number of through holes 13 in the filter portion 12. The thickness of the frame portion 11 may be greater than the thickness of the filter portion 12 so as to enhance the mechanical strength of the sampling filter 10.

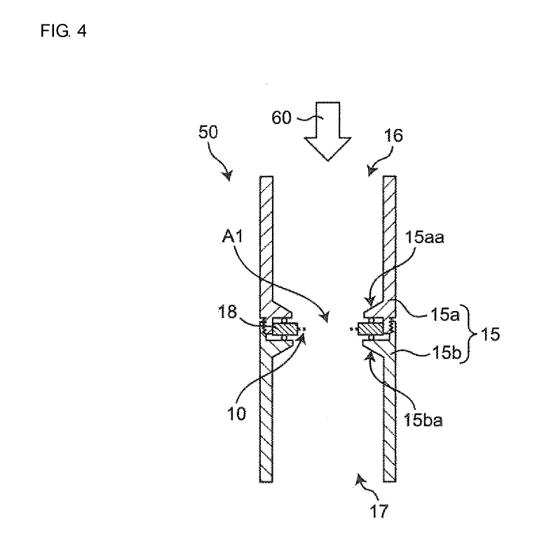

When the sampling filter 10 is connected to a device which supports the sampling filter, the frame portion 11 may function as a connection portion (reference numeral “18” in FIG. 4 described below) that connects the sampling filter 10 to the device. In addition, information relating to the sampling filter 10 (e.g., dimension of the through hole 13 and the like) may be indicated on the frame portion 11.

As noted above, the shape of the frame portion 11 is not limited to a ring as long as the shape demarcates the region A1 through which the liquid passes. The shape of the frame portion 11 may be, for example, a rectangular shape such as a rectangle or a square, or the shape of an ellipse or the like.

<Filter Portion>

As shown in FIGS. 1 and 2, the filter portion 12 is preferably a tabular structure composed of a filter base member 14 (e.g., a sheet of metal or metal oxide) in which a plurality of through holes 13 are located. The filter portion 12 is disposed in a subsection of the region A1 demarcated by the frame portion 11. In the illustrated first embodiment, the filter portion 12 is formed in the shape of a ring whose outer periphery abuts the inner periphery of the frame portion 11. A portion of region A1 radially inward of the filter portion 12 in the vicinity of the center of the region A1. In the preferred embodiment, this region also has through holes but they are larger than the through holes of the filter portion 12 so as to ensure that the flow resistance of the center region is much less than the flow resistance of the filter region 12.

In the illustrated embodiment, the filter portion 12 has a ring shape and is located on the radially outward portion of region A1 adjacent the frame portion 11. However, the invention is not limited to this arrangement. The filter portion 12 can be disposed in any part of the region A1 and it can have a shape other than a ring shape. For example, it can have a rectangular shape such as a rectangle or a square, an elliptical shape or the like. Here, “the filter portion 12 is disposed in part of the region A1 demarcated by the frame portion 11” means that the filter portion 12 is disposed somewhere in the region A1 and that the area of the filter portion 12 (i.e., the area of the principal surface PS2) is less than the area of region A1 in which the filter portion 12 is not disposed.

FIG. 3 is a schematic diagram showing part of the filter portion 12 when viewed along the principal surface PS1. As shown in FIG. 3, the plurality of through holes 13 are preferably periodically arranged on the first and second principal surfaces PS1 and PS2 of the filter portion 12. Specifically, in the illustrated embodiment, the plurality of through holes 13 are located in the matrix at regular intervals in the filter portion 12. In other words, the plurality of through holes 13 are arranged in the shape of a lattice.

In the first embodiment, each through hole 13 has a square shape when viewed along the principal surface PS1. In this regard, the shape of the through hole 13 is not limited to a square shape and may have, for example, a polygonal (e.g., a rectangle), a circular, or an elliptical shape.

In the first embodiment, the shape (cross-sectional shape) of the through hole 13 is a rectangle when projected on a plane perpendicular to the first principal surface PS1 of the filter portion 12. Specifically, the cross-sectional shape of the through hole 13 is a rectangle in which the length of the side in the radius direction of the sampling filter 10 is more than the length of the side in the thickness direction of the sampling filter 10. In this regard, the cross-sectional shape of the through hole 13 is not limited to a rectangle and may be, for example, a parallelogram or a tapered shape such as a trapezoid or be a symmetrical shape or an asymmetrical shape.

In the first embodiment, the plurality of through holes 13 are disposed at regular intervals in two arrangement axes each parallel to a side of a square when viewed along the Z-axis, that is, in the X-axis and the Y-axis. The aperture ratio can be increased by disposing the plurality of through holes 13 in a square lattice arrangement as described above and the passage resistance of the culture medium due to the sampling filter 10 can be reduced. According to such a configuration, stress applied to cell aggregates can be reduced when the liquid passes through the filter portion 12. In addition, the symmetry in the arrangement of the plurality of through holes 13 is improved and, therefore, the filter is readily observed.

In this regard, the arrangement of the plurality of through holes 13 is not limited to the square lattice arrangement and may be, for example, a quasi-periodic arrangement or a periodic arrangement. Examples of the periodic arrangement may include a rectangular arrangement, in which the intervals in the two arrangement axes are not equal, as long as the periodic arrangement is a quadrate arrangement, a triangular lattice arrangement, or a equilateral triangular lattice arrangement. In this regard, a plurality of through holes 13 have to be disposed in the filter portion 12, and there is no limitation regarding the arrangement.

The intervals of the through holes 13 are appropriately designed in accordance with the type (size, form, properties, and elasticity) or the amount of cell aggregates to be separated from the culture medium. As shown in FIG. 3, the interval of the through holes 13 refers to a distance b between the center of any through hole 13 and the center of an adjacent through hole 13 when viewed from the first principal surface PS1 of the filter portion 12. In the first embodiment, the center of the through hole 13 refers to the point of intersection of two diagonals of the square through hole 13. Regarding a periodic arrangement structure, the interval b of the through holes 13 is, for example, more than 1 time and 10 times or less the side d of the through hole 13 and preferably 3 times or less the side d of the through hole 13.

The through holes 13 are designed to have dimensions capable of trapping cell aggregates having a predetermined size. That is, the through holes 13 can trap cell aggregates having a predetermined size in the liquid containing cell aggregates having different sizes. In this regard, the plurality of through holes 13 are designed to have substantially the same dimension. Here, “being designed to have substantially the same dimension” refers to being designed to have dimensions with variations that fall within ±10% or less.

Regarding each through hole 13, the opening in the first principal surface PS1 communicates with the opening in the second principal surface PS2 by a continuous wall surface interposed therebetween. Specifically, the through hole 13 is disposed such that the opening in the first principal surface PS1 can be projected on the opening in the second principal surface PS2. That is, when the filter portion 12 is viewed from the first principal surface PS1, the through hole 13 is disposed such that the opening in the first principal surface PS1 is in accord with the opening in the second principal surface PS2. In the first embodiment, the first through hole 13 is disposed such that the inner wall becomes perpendicular to the first principal surface PS1 and the second principal surface PS2.

The area of the filter portion 12 is preferably, for example, 1% or more and 10% or less than the total area of the region A1 when viewed along the first principal surface of the sampling filter 10. According to such a configuration, an increase in the passage resistance of the liquid containing cell aggregates due to the filter portion 12 can be suppressed.

The thickness of the filter portion 12 (as measured along the Z-axis) is preferably between 0.01 and 10 times the size (a side d) of the through holes 13. The thickness of the filter portion 12 is more preferably between 0.02 and 5 times the size (a side d) of the through holes 13. According to such a configuration, the mechanical strength of the sampling filter 10 can be ensured and, in addition, the passage resistance of the liquid containing cell aggregates can be reduced. As a result, stress applied to cell aggregates can be reduced.

The first principal surface PS1 with which the culture medium containing cell aggregates comes into contact may be flat and smooth. Specifically, the first principal surface PS1 of the filter portion 12 may be formed from a uniform plane with no unevenness. In other words, the openings of the plurality of through holes 13 in the first principal surface PS1 of the filter portion 12 are located on the same plane. The portion of the filter portion 12 in which the through holes 13 is not located, that is, the filter base member portion 14, is continuous and is integrally formed. According to such a configuration, cell aggregates trapped by the through holes 13 can be readily recovered. In addition, observation by a microscope is facilitated because the first principal surface PS1 of the filter portion 12 is formed to become flat and smooth. For example, the sampling filter 10 that has trapped cell aggregates can be brought to the microscope without being processed, and the cell aggregates trapped on the first principal surface PS1 can be observed. At this time, operations of focusing and the like can be readily performed even at a high magnification because the first principal surface PS1 is formed from a uniform plane with no unevenness.

[Sampling Device]

An exemplary sampling device 50 will be described with reference to FIG. 4.

FIG. 4 is a schematic diagram showing the sampling device 50 in the first embodiment according to the present invention. As shown in FIG. 4, the sampling device 50 includes a container portion 15 and the sampling filter 10 arranged inside the container portion 15. In the first embodiment, the sampling filter 10 is connected to the container portion 15 with a connection portion 18 interposed therebetween.

<Container Portion>

The container portion 15 is a tubular body having an inflow port 16 that introduces a liquid containing cell aggregates and an outflow port 17 that discharges the liquid. In the first embodiment, the container portion 15 is a circular-cylindrical tubular body. However, the container portion 15 is not limited to a cylindrical shape as long as flow passages through which the liquid passes are included inside the container portion 15. The shape of the container portion 15 may be, for example, an elliptical or rectangular (including square) when viewed from the inflow port 16.

The container portion 15 can be formed of, for example, a material capable of being gamma sterilized. The container portion 15 may be formed of a material containing, for example, polyethylenes, polyethylene terephthalates, polyurethanes, polystyrenes, silicon rubber, ABS resins, polyamides, polyamide imides, polysulfones, natural rubber, latex, urethane rubber, silicon rubber, ethylene vinyl acetate, polyesters, epoxies, phenols, silica, alumina, gold, platinum, nickel, stainless steel, or titanium. Stress applied to cell aggregates can be reduced by forming the container portion 15 by using such a material.

In the first embodiment, the container portion 15 is composed of first and second member 15a and 15b. The first member 15a is connected to the second member 15b by, for example, complementary male and female screw portions formed at their respective end portions. First and second protrusion portions 15aa and 15ba are formed on the inner walls of the first and second members 15a and member 15b, respectively, so as to hold the frame portion 11 of the sampling filter 10 there between. With this structure, when the first member 15a is connected to the second member 15b, the frame portion 11 of the sampling filter 10 is held between the first protrusion portion 15aa and the second protrusion portion 15ba with the connection portion 18 interposed therebetween. As a result of this configuration, the sampling filter 10 is arranged between the inflow port 16 and the outflow port 17 of the container portion 15. Consequently, when a liquid containing cell aggregates is introduced from the inflow port 16 of the container portion 15, the liquid passes through the sampling filter 10 arranged inside the container portion 15 and, thereafter, is discharged from the outflow port 17.

In the first embodiment of the sampling device 50, the liquid containing cell aggregates preferably passes through the sampling filter 10 under the influence of gravity vertically applied to the first principal surface PS1 of the sampling filter 10 in a downward direction, that is, under the self-weight of the liquid. Alternatively or in addition, the sampling device 50 may include a pressing portion that applies a pressure to the liquid in a direction 60 from the inflow port 16 of the container portion 15 toward the outflow port 17. As a further alternative (or addition), the sampling device 50 may include a suction portion so as to suction the liquid from the outflow port 17 in the direction 60. When the sampling device 50 includes the pressing portion and/or the suction portion, the treatment time can be reduced. In this regard, when pressure and/or suction is/are applied, it is preferable that the force be such an extent that does not deform cell aggregates.

After cell aggregates are sampled by the sampling filter 10 (using the sampling device 50), the first member 15a can be removed from the second member 15b. Subsequently, the sampling filter 10 that has trapped cell aggregates can be set into a microscope while the sampling filter 10 is attached to the second member 15b. Therefore, the cell aggregates trapped by the sampling filter 10 can be readily observed. As a result, the sampling device 50 can readily examine the culture state of the cell aggregates that are trapped by the through holes 13 and that have a predetermined size.

[Sampling Method]

A sampling method using the sampling filter 10 will now be described.

Initially, the sampling filter 10 is prepared (or obtained). The sampling filter 10 is then attached to the container portion 15. In the preferred embodiment, the connection portion 18 is attached to the frame portion 11 of the sampling filter 10 and, thereafter, the sampling filter 10 is arranged on the second protrusion portion 15ba of the second member 15b. Subsequently, the first member 15a is connected to the second member 15b such that the first protrusion portion 15aa of the first member 15a comes into contact with the connection portion 18. In the first embodiment, the first member 15a is connected to the second member 15b by screwing the screw portions. Consequently, the connection portion 18 attached to the frame portion 11 of the sampling filter 10 is held between the first and second protrusion portions 15aa and 15ba.

Then, the liquid containing cell aggregates is passed through the sampling filter 10. More particularly, the liquid flows inside the container portion 15 from the inflow port 16 of the container portion 15 toward the outflow port 17 and, as a result, the liquid flows in the region A1 demarcated by the frame portion 11 of the sampling filter 10. Consequently, part of the liquid containing cell aggregates passes through the filter portion 12 disposed in part of the region A1. At this time, cell aggregates having a predetermined size are trapped by the through holes 13 located in the filter portion 12. On the other hand, the liquid containing cell aggregates passes through the central portion of the region A1 (i.e., the portion of region A1 wherein the filter portion 12 is not disposed) of the filter 10 without undergoing resistance (or at least with significantly lower region that the resistance in filter portion 12).

Examples of methods for passing the liquid containing cell aggregates through the sampling filter 10 include a method in which the liquid is passed substantially vertically from just above the first principal surface PS1 of the filter portion 12 under the action of the gravity. In addition, or alternatively, a pressure can applied to the liquid in a direction 60 from the inflow port 16 of the container portion 15 toward the outflow port 17 and/or the liquid can be suctioned from the second principal surface PS2 in the direction 60. Any combination of those forces can be used individually or together. In any event, it is preferable that stress applied to the cell aggregates be minimized. For example, when a pressure is applied, it is preferable that the pressure be such an extent that does not deform cell aggregates. More preferably, the liquid is passed through the filter portion 12 by the action of the self-weight of the liquid without applying a pressure. Alternatively, it is preferable that the treatment time be reduced by increasing the aperture ratio of the filter portion 12 so as to reduce the time in which stress is applied to cell aggregates.

In the first embodiment, the through holes 13 in the filter portion 12 are designed to have dimensions capable of trapping cell aggregates having a predetermined size. Therefore, part of cell aggregates having a predetermined size can be trapped by the through holes 13 when part of the liquid containing cell aggregates passes through the filter portion 12.

[Effects]

The sampling filter 10 according to the first embodiment can achieve the following effects.

In the sampling filter 10 including the filter portion 12, the region A1 through which the liquid containing cell aggregates passes is demarcated by the frame portion 11, and the plurality of through holes 13 are located in part of the region A1. According to such a configuration, when the liquid containing cell aggregates passes through the region A1, part of the liquid passes through the filter portion 12 and, thereby, cell aggregates having a predetermined size can be readily trapped from the liquid. That is, according to the sampling filter 10, cell aggregates having a predetermined size can be readily sampled without performing filtering a plurality of times.

In the sampling filter 10, the proportion of the area of the filter portion 12 is between 1% and 10%, inclusively, of the area of the region A1 when viewed along the Z-axis. According to such a configuration, 90% or more of the liquid containing cell aggregates passes through the portion that does not include the filter portion 12 in the region A1. On the other hand, the remainder of the liquid passes through the filter portion 12. Consequently, an increase in passage resistance of the liquid due to presence of the filter portion 12 can be suppressed. That is, according to the sampling filter 10, the treatment time can be reduced and, in addition, cell aggregates having a predetermined size can be readily sampled.

The filter portion 12 is disposed so as to have a ring shape abutting the inner wall of the frame portion 11 when viewed along the first principal surface PS1. According to such a configuration, cell aggregates that are targets of sampling are further readily trapped by the filter portion 12. For example, the liquid that flows inside the container portion 15 flows along the inner wall of the container portion 15. Therefore, when the sampling filter 10 is attached inside the container portion 15, the liquid readily passes through the filter portion 12 by disposing the filter portion 12 at a location near the inner wall of the container portion 15. For example, when cell aggregates contained in a liquid having high viscosity are sampled, cell aggregates can be further readily sampled because the liquid tends to flow along the inner wall of the frame portion 11.

A primary component of the sampling filter 10 is at least one of a metal or a metal oxide. According to such a configuration, the mechanical strength of the sampling filter 10 can be enhanced compared with a resin filter such as a membrane. According to the sampling filter 10, for example, when the liquid containing cell aggregates passes through the filter portion 12, the through holes 13 are not readily deformed and, therefore, cell aggregates having a predetermined size are suppressed from passing through the filter portion 12.

The sampling device 50 and the sampling method can also exert the same effects as the above-described effects of the sampling filter 10. In particular, the handleability of the sampling filter 10 can be improved by using the sampling device 50. For example, the cell aggregates trapped on the first principal surface PS1 of the sampling filter 10 can be readily observed while the sampling filter 10 is attached to the second member 15b of the container portion 15.

In the first embodiment, the configuration in which a primary component of the sampling filter 10 is at least one of a metal or a metal oxide is described, but the configuration is not limited to this. The sampling filter 10 has to be a porous film and may be, for example, nylon, polypropylene, polyethylene, polyester, polyether ether ketone, polyethylene terephthalate, or polyvinylidene fluoride.

In the first embodiment, the example in which the sampling filter 10 traps cell aggregates having a predetermined size is described, but the sampling filter 10 is not limited to this. The sampling filter 10 may trap an object other than cell aggregates. That is, the sampling filter 10 may be used for obtaining the information on presence of an object contained in the liquid by trapping the object present in the liquid. The sampling filter 10 may be used for obtaining the information on presence of, for example, single cells, dead cells, and resin beads.

In the first embodiment, the example in which the filter portion 12 is disposed so as to have the shape of a ring along the inner wall of the frame portion 11 is described, but the filter portion 12 is not limited to this. The filter portion 12 has to be disposed in part of the region A1 demarcated by the frame portion 11 and its shape is not limited. The filter portion 12 can be designed in accordance with the flow of the liquid that passes through the region A1. For example, the filter portion 12 may be disposed at a location at which the liquid concentratedly flows in the region A1 or be disposed at a location at which the flow of the liquid is not hindered as much as possible in the region A1.

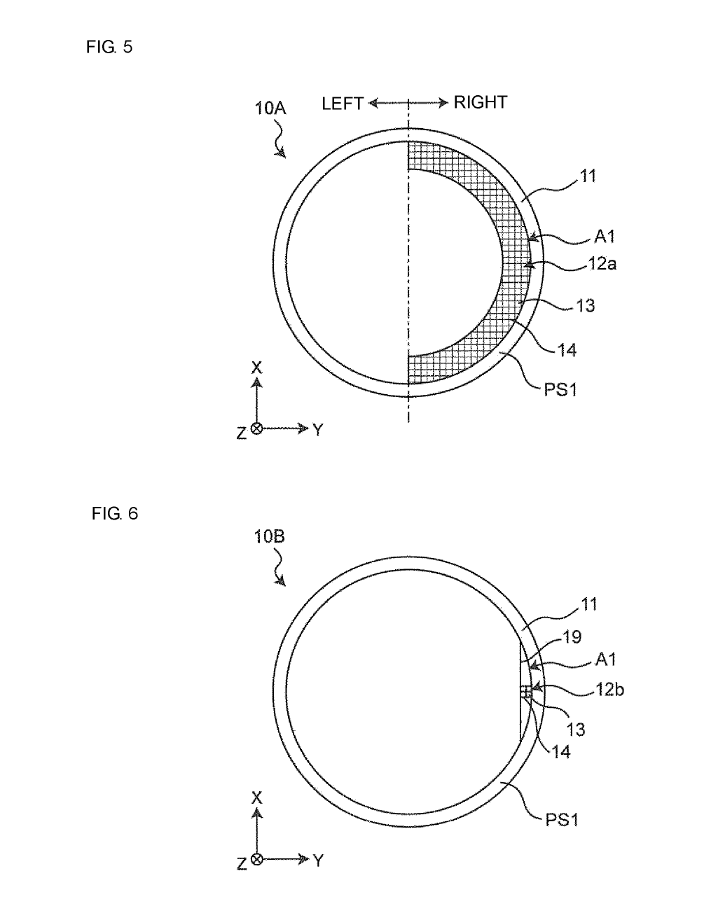

FIG. 5 is a schematic diagram showing a sampling filter 10A in a modified example of the first embodiment according to the present invention. As shown in FIG. 5, in sampling filter 10A, a filter portion 12a may be disposed so as to have the shape of, for example, an arch along part of the inner wall of the frame portion 11. In other words, the sampling filter 10A may includes the filter portion 12a having a curved shape along the right inner wall of the frame portion 11 (as viewed in FIG. 5) and may include no filter portion 12a along the left inner wall of the frame portion 11, when viewed along the first principal surface PS1. The filter portion 12a in the region A1 is not limited to the example shown in FIG. 5 and, for example, may be disposed along the left inner wall of the frame portion 11 when viewed from the first principal surface PS1. The proportion of the area of the filter portion 12a relative to the total area of the region A1, when viewed along the first principal surface PS1, may be changed in accordance with the number of cell aggregates to be sampled.

FIG. 6 is a schematic diagram showing a sampling filter 10B in another modified example of the first embodiment according to the present invention. As shown in FIG. 6, in the sampling filter 10B, a filter portion 12b may be held by a holding portion 19. The holding portion 19 extends from the inner wall of the frame portion 11 and holds the filter portion 12b. The holding portion 19 is composed of, for example, the same material as the filter base member portion 14. According to such a configuration, the mechanical strength of the filter portion 12b can be enhanced. In this regard, the holding portion 19 may be integrally formed with the filter base member portion 14 and the frame portion 11 of the filter portion 12b. The mechanical strength can be further enhanced by integrally forming the holding portion 19 with the filter base member portion 14 and the frame portion 11.

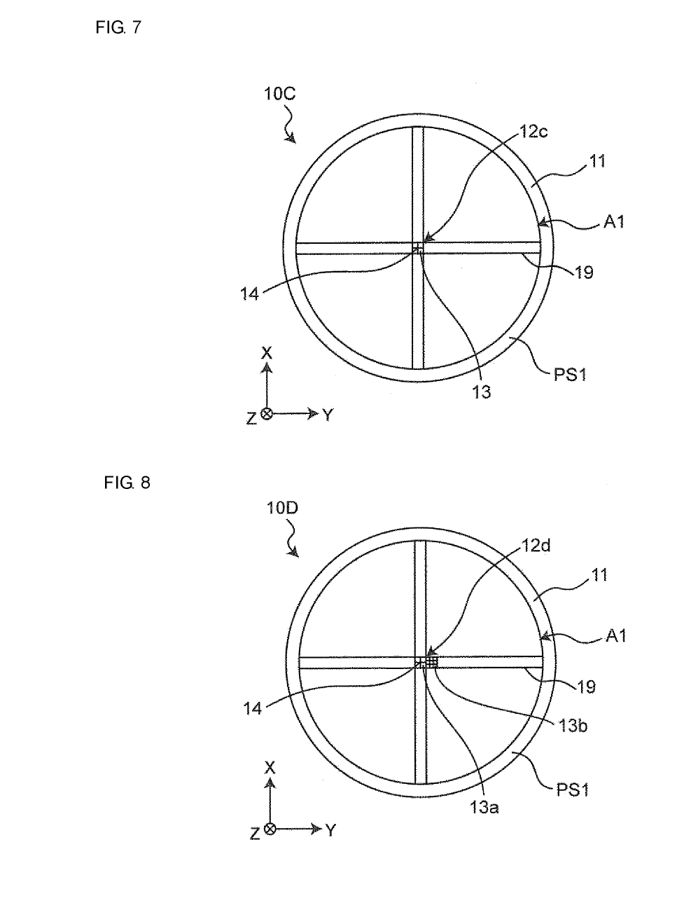

FIG. 7 is a schematic diagram showing a sampling filter 10C in another modified example of the first embodiment according to the present invention. As shown in FIG. 7, in the sampling filter 10C, a filter portion 12c may be disposed at the center of the region A1, when viewed from the first principal surface PS1, by being held by a plurality of holding portions 19. Specifically, two holding portions 19 that extend along the Y-axis from the inner wall of the frame portion 11 and two holding portions 19 that extend along the X-axis from the inner wall of the frame portion 11 are disposed so as to intersect with each other at the center of the region A1 as shown in FIG. 7. Then, the filter portion 12c is held by a radially inner portion of the holding portions that intersect with each other. More generally, the filter portion 12c can be disposed at any location in the region A1 by using appropriately designed holding portions 19. According to such a configuration, the filter portion 12c can be disposed at a location at which sampling is readily performed or be disposed at a location at which the flow of the liquid is not readily hindered, in accordance with the flow of the liquid.

FIG. 8 is a schematic diagram showing a sampling filter 10D in another modified example of the first embodiment according to the present invention. In the sampling filter 10D shown in FIG. 8, in addition to the configuration of the sampling filter 10C shown in FIG. 7, through holes 13a and 13b having different sizes are located in a filter portion 12d. As shown in FIG. 8, a plurality of first through holes 13a and a plurality of through holes 13b smaller than the first through holes 13a are located in the filter portion 12d. According to such a configuration, cell aggregates having different sizes can be trapped by performing sampling once. In this regard, in the filter portion 12d, the through holes 13a and 13d are not limited to two sizes, and through holes having at least two sizes may be located.

The above-described sampling filters 10A, 10B, 10C, and 10D are exemplifications, and the sampling filter 10 according to the present invention is not limited to these examples. It is sufficient for the sampling filter 10 to include a filter portion 12 in which through holes 13 that trap cell aggregates are located in part of the region A1 through which the liquid containing cell aggregates passes. The location at which the filter portion 12 is located, the area (size and/or shape) if the filter portion 12, and the like may be appropriately designed in accordance with the number of cell aggregates to be sampled, the flow of the liquid, and the like.

In the first embodiment, the example in which the container portion 15 of the sampling device 50 is composed of the two members, that is, the first member 15a and the second member 15b, is described, but the configuration is not limited to this. Meanwhile, in the first embodiment, the example in which the first member 15a is connected to the second member 15b by screwing the screw portions is described, but the configuration is not limited to this. For example, in the container portion 15, the first member 15a and the second member 15b may be integrally formed. According to such a configuration, the strength of the container portion 15 can be enhanced.

In the first embodiment, the example in which the sampling filter 10 in the sampling device 50 is arranged between the inflow port 16 and the outflow port 17 of the container portion 15 is described, but the configuration is not limited to this. It is sufficient that the sampling filter 10 is arranged at a location through which the liquid containing cell aggregates can pass. For example, the sampling filter 10 may be arranged in the inflow port 16 of the container portion 15 or be arranged in the outflow port 17 of the container portion 15.

In the first embodiment, the example in which the sampling device 50 includes one sampling filter 10 is described, but the configuration is not limited to this. The sampling device 50 may include a plurality of sampling filters 10. When the sampling device 50 includes a plurality of sampling filters 10, the number of cell aggregates having predetermined size that can be sampled is increased. For example, if it is desired that the proportion of the area of the filter portion 12 relative to the area of the region A1 be decreased (when viewed along the first principal surface PS1), a plurality of sampling filters 10 can be arranged in stages (i.e., one after the other along the flow direction). Consequently, the number of cell aggregates sampled can be increased while an increase in passage resistance of the liquid due to the sampling filter 10 is suppressed. In addition, a plurality of sampling filters 10 having different sized through holes 13 may be included. With such a configuration, cell aggregates having different sizes can be sampled by performing sampling once.

Regarding the sampling method, the number of cell aggregates sampled may be increased or cell aggregates having different sizes may be sampled by passing the liquid containing cell aggregates through a plurality of sampling filters 10, as described above.

### Second Embodiment

[Sampling Device]

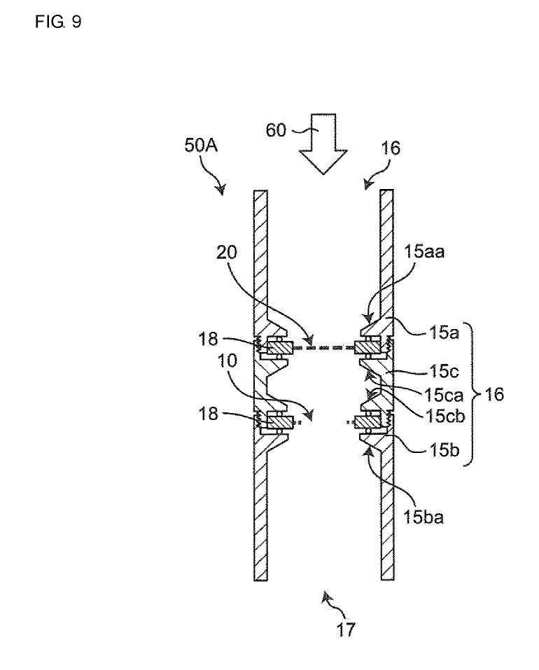

A sampling device in a second embodiment according to the present invention will be described with reference to FIG. 9. FIG. 9 is a schematic diagram showing a sampling device 50A in the second embodiment according to the present invention.

In the second embodiment, points of difference from the first embodiment will be mainly described. In explanations of the second embodiment, the same configurations as in the first embodiment are indicated by the same reference numerals as those set forth in the first embodiment. In this regard, explanations in the first embodiment are not repeated in the second embodiment.

As shown in FIG. 9, the sampling device 50A in the second embodiment is different from the first embodiment in that a filtration filter 20 is included upstream of the sampling filter 10 and that the container portion 15 is composed of three members 15a, 15b, and 15c.

In the sampling device 50A, first cell aggregates having a size greater than the predetermined size are removed from the liquid containing cell aggregates by passing the liquid through the filtration filter 20. Subsequently, the liquid is passed through the sampling filter 10 and, thereby, second cell aggregates smaller than the first cell aggregates are sampled.

<Filtration Filter>

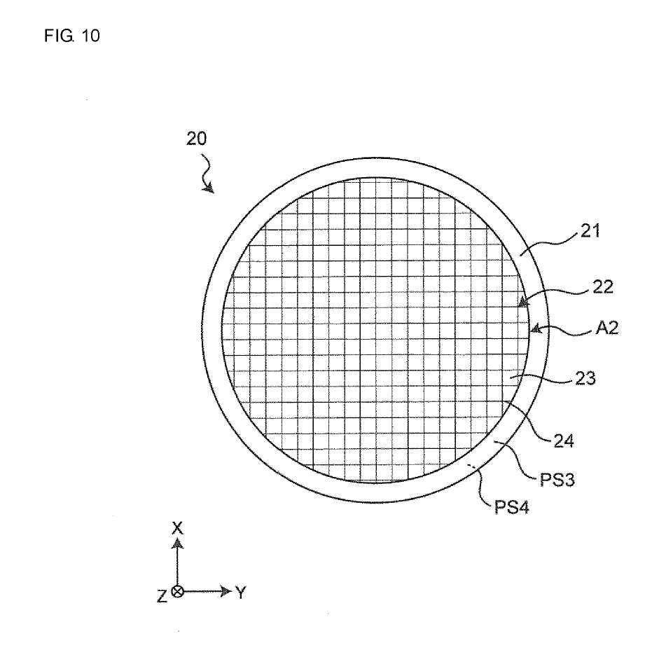

FIG. 10 is a schematic diagram showing the filtration filter 20 used in the sampling device 50A in the second embodiment according to the present invention. In FIG. 10, X-axis, Y-axis, and Z-axis indicate the vertical direction, the horizontal direction, and the thickness direction, respectively, of the filtration filter 20.

As shown in FIG. 10, the filtration filter 20 is preferably a circular metal mesh. The filtration filter 20 includes a frame portion 21 that demarcates a region A2 through which the liquid containing cell aggregates passes and a filter portion 22 disposed in the region A2. Specifically, the frame portion 21 is disposed on the outer perimeter of the filter portion 22. The filter portion 22 includes a first principal surface PS3 and a second principal surface PS4 opposite to each other. The filter portion 22 includes a filter base member portion 24 in which a plurality of through holes 23 passing through the first principal surface PS3 and the second principal surface PS4 are located. The through holes 23 in the filtration filter 20 are designed to be larger than the through holes 13 in the sampling filter 10. The filtration filter 20 includes the filter portion 22 in the entire region A2 that is demarcated by the frame portion 21 when viewed along the first principal surface PS3.

In the second embodiment, the filtration filter 20 has the same configuration as the sampling filter 10 except that it does not include the filter portion 12. In other words, the filtration filter 20 is different from the sampling filter 10 in that the filter portion 22 is disposed in the entire region A2 of the frame portion 21 and that the through holes 23 in the filtration filter 20 are designed to have a larger size than the through holes 13 in the sampling filter 10.

<Container Portion>

As shown in FIG. 9, the container portion 15 includes a first member 15a, a second member 15b, and a third member 15c. That is, the container portion 15 of the sampling device 50A in the second embodiment is different from the container portion 15 in the first embodiment in that the third member 15c is included.

The third member 15c is connected between the first and second members 15a and 15b. Like the first and second member, the third member 15c is preferably a circular-cylindrical tubular body. Screw portions are disposed at opposite ends of the third member 15c. One end portion of the third member 15c and the first member 15a are screwed together, and the other end portion and the second member 15b are screwed together. A third protrusion portion 15ca so as to hold the filtration filter 20 and a fourth protrusion portion 15cb so as to hold the sampling filter 10 are formed on the inner wall of the third member 15c. Specifically, when the first member 15a is connected to the third member 15c, the connection portion 18 attached to the frame portion 21 of the filtration filter 20 is held between the first protrusion portion 15aa of the first member 15a and the third protrusion portion 15ca of the third member 15c. Meanwhile, when the second member 15b is connected to the third member 15c, the connection portion 18 attached to the frame portion 11 of the sampling filter 10 is held between the second protrusion portion 15ba of the second member 15b and the fourth protrusion portion 15cb of the third member. According to such a configuration, the filtration filter 20 and the sampling filter 10 can be arranged between the inflow port 16 and the outflow port 17 of the container portion 15 and, in addition, the filtration filter 20 can be arranged nearer than the sampling filter 10 to the inflow port 16.

[Sampling Method]

Next, a sampling method in the second embodiment according to the present invention will be described.

The sampling method in the second embodiment includes placing the filtration filter 20 upstream of the sampling filter 10 and trapping the first cell aggregates by first passing the liquid containing cell aggregates through the filtration filter 20 and then through the sampling filter 10. Consequently, first cell aggregates that are larger than the predetermined size can be removed from the liquid containing cell aggregates having different sizes by the filtration filter 20.

Second cell aggregates that are smaller than the first cell aggregates pass through the filtration filter 10 and are trapped by passing the liquid filtrated by the filtration filter 20 through the sampling filter 10. Specifically, the second cell aggregates having a predetermined size are trapped by the through holes 13 by passing part of the liquid from which the first cell aggregates have been removed through the filter portion 12 of the sampling filter 10. As a result, the second cell aggregates having a predetermined size can be sampled.

[Effects]

The sampling device 50A according to the second embodiment can exert the following effects.

The sampling device 50A includes the filtration filter 20 arranged upstream of the sampling filter 10. The filtration filter 20 traps the first cell aggregates larger than the predetermined size from the liquid containing cell aggregates having different sizes. Meanwhile, the sampling filter 10 traps the second cell aggregates having a predetermined size in the liquid from which the first cell aggregates have been removed by the filtration filter 20. According to such a configuration, the second cell aggregates having a predetermined size can be sampled while the first cell aggregates larger than the predetermined size are filtrated.

The sampling method can also exert the same effects as the above-described effects of the sampling device 50A. In particular, when filtering is performed by using the filtration filter 20 for the purpose of obtaining cell aggregates having a predetermined size, the cell aggregates having a predetermined size can be sampled by the sampling filter 10. Consequently, according to the sampling method of the present invention, cell aggregates having a predetermined size can be readily sampled from the liquid containing cell aggregates having different sizes without performing filtering a plurality of times.

In this regard, in the second embodiment, the example in which the filtration filter 20 is the circular metal mesh is described, but the configuration is not limited to this. The filtration filter 20 has to be a filter capable of trapping the first cell aggregates larger than the predetermined size from the liquid containing cell aggregates having different sizes. The filtration filter 20 may be a filter such as a membrane or a resin mesh. Meanwhile, the design of the dimension and the shape of the filtration filter 20 can be appropriately changed in accordance with, for example, the shape of the container portion 15.

In the second embodiment, the example in which the sampling device 50A includes only one filtration filter 20 is described, but the configuration is not limited to this. The sampling device 50A may include a plurality of filtration filters 20. Likewise, the example in which the sampling device 50A includes only one sampling filter 10 is described, but the configuration is not limited to this. The sampling device 50A may include a plurality of sampling filters 10.

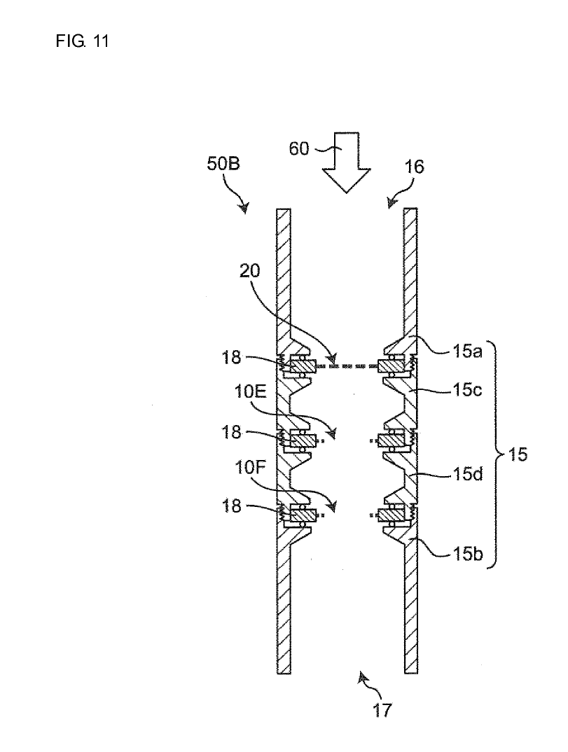

FIG. 11 shows a sampling device 50B in a modified example of the second embodiment according to the present invention. As shown in FIG. 11, the sampling device 50B includes two sampling filters 10E and 10F located downstream of the filtration filter 20.

Specifically, a first sampling filter 10E is arranged downstream of the filtration filter 20, and a second sampling filter 10F is arranged downstream of the first sampling filter 10E. The first sampling filter 10E and the second sampling filter 10F may be the same or different than one another.

When the first sampling filter 10E and the second sampling filter 10F are the same, the number of cell aggregates having a predetermined size sampled can be increased.

When the first sampling filter 10E and the second sampling filter 10F are different filters, each of filters can sample cell aggregates having sizes different on a filter basis. In the sampling device 50B, for example, through holes smaller than the through holes 13 in the first sampling filter 10E may be located in the second sampling filter 10F. According to such a configuration, the first cell aggregates larger than the predetermined size can be removed by the filtration filter 20, the second cell aggregates having the predetermined size can be extracted by the first sampling filter 10E, and the third cell aggregates smaller than the second cell aggregates can be extracted by the second sampling filter 10F.

Meanwhile, the container portion 15 of the sampling device 50B may be composed of four members, that is, a first member 15a, a second member 15b, a third member 15c, and a fourth member 15d. The fourth member 15d may be composed of the same member as the third member 15c.

In the second embodiment, the example in which the filtration filter 20 traps the first cell aggregates and the sampling filter 10 traps the second cell aggregates is described, but the configuration is not limited to this. The filtration filter 20 and the sampling filter 10 may trap objects other than cell aggregates. For example, the filtration filter 20 may traps cell membranes. Meanwhile, the sampling filter 10 may be used for obtaining the information on presence of an object contained in the liquid by trapping the object other than the cell aggregates that has passed through the filtration filter 20.

The present invention has been described in detail with reference to the preferred embodiments and the accompanying drawings. It is obvious that various changes and modifications thereof can be made by one skilled in the art. It should be understood that such changes and modifications made without departing from the scope of the invention as defined in the appended claims are included in the present invention.

### REFERENCE SIGNS LIST

10, 10A, 10B, 10C, 10D, 10E, 10F sampling filter

11 frame portion

12 filter portion

13 through hole

14 filter base member portion

15 container portion

15a first member

15aa first protrusion portion

15b second member

15ba second protrusion portion

15c third member

15ca third protrusion portion

15cb fourth protrusion portion

15d fourth member

16 inflow port

17 outflow port

18 connection portion

20 filtration filter

21 frame portion

22 filter portion

23 through hole

24 filter base member portion

50, 50A, 50B sampling device

60 direction

PS1 first principal surface

PS2 second principal surface

PS3 first principal surface

PS4 second principal surface

1. A sampling filter that samples cell aggregates comprising:
a frame portion that demarcates a region through which a liquid containing cell aggregates passes; and
a filter portion which is disposed in only part of the region and in which a plurality of through holes are located.

2. The sampling filter according to claim 1, wherein the proportion of the area of the filter portion is between 1% and 10% of the area of the region.

3. The sampling filter according to claim 1, wherein the plurality of through holes in the filter portion include first and second through holes, the second through holes being smaller than the first through holes.

4. The sampling filter according to claim 1, wherein the filter portion is disposed along an inner wall of the frame portion.

5. The sampling filter according to claim 1, further comprising a holding portion that extends from the frame portion and that holds the filter portion.

6. The sampling filter according to claim 1, wherein a primary component of the sampling filter is at least one of a metal or a metal oxide.

7. The sampling filter according to claim 1, wherein the region includes a base portion through which a fluid can flow and the base portion includes the filter portion and a second portion, both of which have through holes, the flow resistance of the filter portion being greater than the flow resistance of the second portion.

8. A sampling device comprising:
a container portion having an inflow port through which a liquid containing cell aggregates can be introduced and an outflow port through which the liquid can be discharged; and
a sampling filter arranged between the inflow port and the outflow port of the container portion, the sampling filter including:
a frame portion that demarcates a region through which the liquid containing cell aggregates passes, and
a filter portion which is disposed in only part of the region and in which a plurality of through holes are located.

9. The sampling device according to claim 8, wherein the proportion of the area of the filter portion is between 1% and 10% of the area of the region.

10. The sampling device according to claim 8, wherein the plurality of through holes in the filter portion include first and second through holes, the second through holes being smaller than the first through holes.

11. The sampling device according to claim 8, wherein the filter portion is disposed along an inner wall of the frame portion.

12. The sampling device according to claim 8, further comprising a holding portion that extends from the frame portion and that holds the filter portion.

13. The sampling device according to claim 8, wherein a primary component of the sampling filter is at least one of a metal or a metal oxide.

14. The sampling device according to claim 8, further comprising a filtration filter which is nearer than the sampling filter to the inflow port of the container portion and in which a plurality of through holes are located,
wherein the through holes in the filtration filter are larger than the through holes in the sampling filter.

15. The sampling device according to claim 13, wherein:
the filtration filter traps first cell aggregates, and
the sampling filter traps second cell aggregates which are smaller than the first cell aggregates.

16. The sampling device according to claim 1, wherein the sampling devices includes a plurality of sampling filters, the plurality of sampling filters including:
a first sampling filter arranged between the inflow port and the outflow port of the container portion and
a second sampling filter arranged nearer than the first sampling filter to the outflow port of the container portion, and
third through holes smaller than the first through holes in the first sampling filter are located in the second sampling filter.

17. The sampling device according to claim 8, wherein the region includes a base portion through which a fluid can flow and the base portion includes the filter portion and a second portion, both of which have through holes, the flow resistance of the filter portion being greater than the flow resistance of the second portion.

18. A sampling method comprising:
obtaining a sampling filter including a frame portion that demarcates a region through which a liquid containing a first set of cell aggregates passes and a filter portion which is disposed in only a part of the region and in which a plurality of through holes are located; and
passing the liquid containing cell aggregates through the sampling filter.

19. The sampling method according to claim 18, wherein the liquid containing the first set of cell aggregates is obtained by first passing a liquid containing by the first and a second set of cell aggregates through a filtration filter including a plurality of through holes through which the liquid can pass so as to trap second cell aggregates, the second set of cell aggregates being larger than the first set of cell aggregates.

20. The sampling method according to claim 19, wherein the sampling filter is a first sampling filter and when the liquid passes through the first sampling filter, the first cell aggregates are trapped on the first sampling filter and third cell aggregates, which are smaller than the second cell aggregates are passed through the first sampling filter, the sampling method further comprising passing the liquid that has passed through the first sampling filter through a second sampling filter and trapping the third cell aggregates on the second sampling filter.
